(19) United States
(12) Patent Application Publication
(10) Pub. No.: US 2019/0192302 A1
(43) Pub. Date: Jun. 27, 2019

(54) PROSTHESIS FOR A TEMPOROMANDIBULAR JOINT

(71) Applicant: CADSKILLS BVBA, Sint-Martens Latem (BE)

(72) Inventor: Maurice Yves MOMMAERTS, Sint-Martens Latem (BE)

(73) Assignee: CADSKILLS BVBA, Sint-Martens Latem (BE)

(21) Appl. No.: 16/301,364

(22) PCT Filed: May 11, 2017
(86) PCT No.: PCT/IB2017/000560

§ 371 Date: Nov. 13, 2018

(30) Foreign Application Priority Data
May 12, 2016 (NL) 1041862

(51) Int. Cl. A61F 2/30

(52) U.S. Cl. CPC A61F 2/3099; A61F 2002/30991; A61F 2/30942; A61F 2002/30131; A61F 2002/30301

(57) ABSTRACT

Prosthesis (1) for the mandibular side of a temporomandibular joint, comprising a head part (2) to replace the condylar head, in particular at the anatomic location, and an attachment plate, wherein the attachment plate is saddle-shaped having a lateral portion (3) and a medial portion (4) that have been formed and are intended for abutting both sides of the ascending branch of the mandible, straight below the arcuate notch in the upper end of the ascending branch of the mandible.

### BACKGROUND OF THE INVENTION

The invention relates to a jaw joint prosthesis, or in other words a prosthesis to replace a temporomandibular joint (TMJ).

In a jaw joint and the area around it, the consecutive area of the mandible (mandibula) and the temporal bone (os temporale) can be distinguished; and in particular as regards the mandible, the condylar process, having a neck and (condylar) head (caput mandibulae); and as regards the temporal bone, the glenoid cavity (fossa glenoidalis), formed behind the zygomatic arc (arcus zygomaticus), including the muscle tissues and interstitial tissues connecting them to each other and the articular disc (discus articularis).

Patients suffering considerable damage to the jaw joint resulting from ankylosis, rheumatoid arthritis, osteoarthritis, tumors and trauma have problems chewing and speech problems. Overcoming these problems may require partial or full replacement of the jaw joint.

From U.S. Pat. No. 3,178,728 a one-sided prosthesis is known. It is formed by a chrome-cobalt casting for covering the glenoid cavity, including the anterior articular tubercle (tuberculum articulare), in order to provide a bearing for the condylar head. A comparable, yet shorter one-sided prosthesis is known from U.S. Pat. No. 3,579,643. This prosthesis leaves the glenoid cavity free. A more recent suggestion is described in US 2009/0222102.

Two-sided prostheses are known among others from U.S. Pat. Nos. 4,693,722, 4,778,472, 4,917,701, 5,549,680, 5,989,292 and French patent application 2,558,721.

A more recent design of a two-sided prosthesis is the subject of U.S. Pat. No. 6,132,466, comprising a replacement of the condylar head and a bearing for it. The condylar head prosthesis comprises a downwardly extending strip to be attached by means of screws to and below the neck of the condylar process. A comparable design is the subject of US patent application 2009/0138092.

Another two-sided prosthesis is known from WO 2014/023903, wherein the portion that is to replace the condylar head is two-part, having a slightly curved pin including prosthetic condylar head which is to be attached in the jaw bone, and a retainer part comprising a downwardly extending strip to be attached by means of screws to and below the neck of the condylar process and a retaining ring integrally formed therewith for keeping the pin in the correct orientation.

### SUMMARY OF THE INVENTION

It is an object of the invention to provide a prosthesis for a condylar head in a temporomandibular joint, which in many cases can be arranged with fewer risks to the patient.

It is an object of the invention to provide a prosthesis for a condylar head in a temporomandibular joint, which in many cases requires less effort to arrange.

It is an object of the invention to provide a two-sided prosthesis for a temporomandibular joint.

According to one aspect the invention provides a prosthesis for the mandibular side of the temporomandibular joint, comprising a head part to replace the condylar head at the anatomic location, and an attachment plate for attachment to the mandible, wherein the attachment plate has been formed having a lateral portion that has been formed and is intended for abutting the lateral side of the ascending branch of the mandible, straight below the arcuate notch (incisura semilunaris) in the upper end of the ascending branch of the mandible, wherein the lateral portion has been provided with holes for attachment to the lateral side of the ascending branch of the mandible and wherein the attachment plate comprises an upper portion forming one unity with the lateral portion and formed and intended for abutting the concave upper edge of the arcuate notch in the upper end of the ascending branch of the mandible.

In that way, a relocation of the locations of transfer of forces exerted on the condylar head, situated at a location corresponding to the anatomic location, to the mandible can be realized, from the areas below the condylar neck known in the art, often to near the jaw angle, to areas situated higher and more to the fore at the ascending branch of the mandible, at the upper edge of the arcuate mandibular notch and below it on the lateral side of the ascending branch of the mandible. By repositioning the attachment area at the ascending branch of the mandible, it is possible to leave the area near the jaw angle undisturbed when placing the prosthesis, because the vertical downward extension can remain within limits, as a result of which it is possible to stay out of the area of the buccal branch of the facial nerve as well. Vertical forces do not only need to be transferred via shear-loaded bone screws, but can also be transferred to the mandible via the support on the naturally provided arcuate upper edge of the said notch. The abutment in vertical direction and in medial direction to the upper edge of the arcuate notch and the lateral side of the mandible, respectively, may facilitate the placement.

In a further development thereof, the attachment plate comprises a medial portion forming one unity with the lateral portion and the upper portion, and having been formed and being intended for abutting the medial side of the ascending branch of the mandible, preferably straight below the arcuate notch in the upper end of the ascending branch of the mandible. In that way, the stability in lateral direction is enhanced. Furthermore, it facilitates the placement of the prosthesis.

In a further development the upper portion, when considered in a direction parallel to dorsal-ventral, has been formed at least partially concave, with the cavity facing upwards.

According to another aspect, the invention provides a prosthesis for the mandibular side of a temporomandibular joint, comprising a head part to replace the condylar head at the anatomic location and an attachment plate, wherein the attachment plate comprises a lateral portion and a medial portion that have been formed and are intended for abutting both sides of the ascending branch of the mandible, straight below the arcuate notch in the upper end of the ascending branch of the mandible, and having an upper portion forming one unity therewith, being formed concave with the cavity facing upwards, when considered in a direction transverse to the lateral portion, and being formed concave, with the cavity facing downwards, when considered in a cross-section. In one embodiment, the attachment plate has the look of a horse saddle, or the look of a hyperbolic paraboloid. The attachment plate may have a double concave shape, both concave shapes being oriented in an opposing manner (upwards and downwards) and rotated over approximately 90 degrees relative to each other.

In one embodiment, only the lateral portion has been provided with holes for attachment to the lateral side of the ascending branch of the mandible.

Dorsal, ventral, lateral, medial, top, bottom, below, etc. refer to the position in the condition of use, placed in a patient who is standing upright.

As noted, the extension of the lateral portion/wing can remain limited in downward direction, in particular in a direction parallel to the direction of projection of the head part, the lateral portion may have a length that is smaller than its length in a (ventral-dorsal) direction transverse thereto.

The shape of the lateral portion or lateral wing will be adapted to the anatomy of the bone. The lateral portion may for instance be sickle-shaped, for instance comprise an L-shaped lip, or comprise several lips, for instance one lip near the condylar neck and one lip near the coronoid process. This list is by no means exhaustive and other shapes are also possible.

The shape of the medial portion or medial wing will be adapted to the anatomy of the bone. With its upper portion, the medial portion may form an overhanging edge abutting the bone. The medial portion may for instance be sickle-shaped, for instance comprise an L-shaped lip, or comprise several lips, for instance one lip near the condylar neck and one lip near the coronoid process. This list is by no means exhaustive and other shapes are also possible.

In ridge direction, the upper portion may be continuous or comprise several portions in which case it is interrupted. The length in ridge direction may be in the order of magnitude of the intermediate distance between the necks of both processes, or a fraction (for instance ½ or ⅓) thereof.

In one development, the lateral portion has been configured for in the placed condition extending in the ventral direction to beyond the lowest point of the arcuate upper edge, that means up into the area situated straight below the portion of the said upper edge which is ascending in the ventral direction (towards the coronoid process).

In one development, the upper portion has been configured for in the placed condition extending in the ventral direction to beyond the lowest point of the arcuate upper edge, that means up into the area of the said upper edge which is ascending in the ventral direction (towards the coronoid process).

In one development, the medial portion has been configured for in the placed condition extending in the ventral direction to beyond the lowest point of the arcuate upper edge, that means up into the area situated straight below the portion of the said upper edge which is ascending in the ventral direction (towards the coronoid process).

If so desired the lateral portion may have been formed and be intended for extending up to the coronoid process. This may for instance be desired in connection with the number of screws to be placed. The extension in frontal direction does not conflict with nerve bundles. The medial portion as well may have been formed and be intended for extending up to the coronoid branch. The medial portion may remain free from the sphenomandibular ligament.

There is also room for the upper portion, and it may have been formed and be intended for extending up to the coronoid process.

In one embodiment, the head part and the attachment plate form one unity with each other.

The prosthesis may have been manufactured, customized to the patient in question. The shape and condition of the ascending branch of the maxilla, in particular its coronoid process and the condylar neck, can be determined by means of scanning. On the basis thereof, a prosthesis that is optimal for the patient in question, can be designed and recorded in a CAD file. Using said file, a computer operated device will be able to make the prosthesis, in a machining action, such as milling, or in an additional production technique, such as injection molding, laser sintering, stereo lithography, in particular FDM, SLM, SLS or another 3D printing technique, for instance of titanium or PEEK (polyether ether ketone). The condylar head may have been provided with a titanium-nitride layer or a thin ceramic layer.

According to a further aspect, the invention provides an assembly of a prosthesis according to the invention and a prosthesis for the fossa glenoidalis as bearing part of the head part. The bearing part allows rotation of the condylar head about the medial-lateral axis as well as translation thereof in antero-posterior (ventral-dorsal) direction, in keeping with a natural situation.

According to a further aspect, the invention provides a method for replacing the condylar head in a patient by a condylar head prosthesis, wherein the shape of the edge of the arcuate notch in the upper end of the ascending branch of the mandible is defined and the prosthesis according to the invention, is also designed and manufactured on the basis thereof, after which the prosthesis is being placed on the ascending branch of the mandible in question.

It is noted that a two-sided TMJ prosthesis is known from JP H 11 146889, wherein in one embodiment the condylar head is replaced at the anatomic location and the attachment to the lateral side of the ascending branch of the mandible takes place using an attachment strip extending up into the area of the jaw angle.

From U.S. Pat. No. 4,726,808 a mandibular component is known having a prosthesis for the condylar head of a TMJ, which is provided with two attachment strips to be attached to the lateral and medial sides of the ascending branch of the mandible, which strips extend up into the area of the mandible, while leaving the edge of the incisura semilunaris free.

The aspects and measures described in this description and the claims of the application and/or shown in the drawings of this application may where possible also be used individually. Said individual aspects may be the subject of divisional patent applications relating thereto. This particularly applies to the measures and aspects that are described per se in the sub claims.

### BRIEF DESCRIPTION OF THE DRAWINGS

The invention will be elucidated on the basis of an exemplary embodiment shown in the attached drawings. The views are named in accordance with the side in question, that means that a lateral view is a view from the lateral side. In which:

FIGS. 1A-E show a medial side view, a lateral side view, a postera-lateral view, a postera-medial view and a view in cross-section according to plane IE in FIG. 1B, respectively, of the mandibular component of the jaw joint prosthesis to be placed on a mandible;

FIGS. 2A and 2B show a postera-lateral view and a postera-medial view, respectively, of a fossa component of a jaw joint prosthesis to be placed on a maxilla;

FIGS. 3A and 3B show a postera-lateral view and a postera-medial view of an assembly of the mandibular component of FIGS. 1A-E and the fossa component of FIGS. 2A, B; and

FIGS. 4A and 4B show a lateral and a medial side view, respectively, of an assembly according to the invention in the placed condition.

### DETAILED DESCRIPTION OF THE DRAWINGS

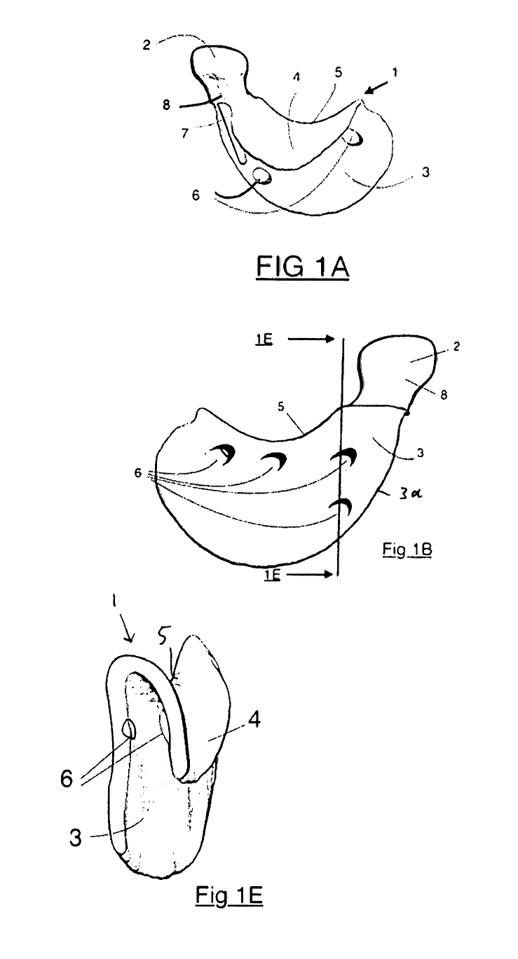

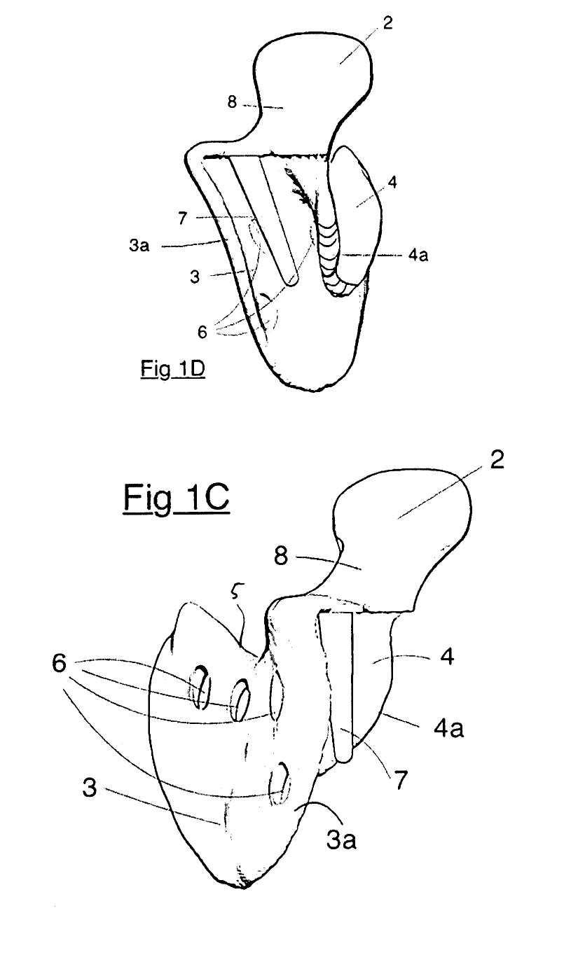

FIGS. 1A-D show the mandibular component 1 of a temporomandibular joint prosthesis (TMJ prosthesis), which comprises a lateral wing 3 and a medial wing 4, that are connected to each other by an upper portion or ridge 5 forming one unity therewith. The lateral wing 3 has been provided with bone screw holes 6. It is clear to see that the component 1 is saddle-shaped, has an inverted U-shape in ventral and dorsal view, and is U-shaped in side view (when considered transverse to the lateral portion, see FIG. 1D). The attachment plate in this case has a double concave shape, both concave shapes being oriented in an opposing manner (upwards and downwards) and rotated over approximately 90 degrees relative to each other. The attachment plate may also have the look of a hyperbolic paraboloid.

At the rear end via a neck 8, an artificial condylar head 2 projects upwards, like a kind of saddle horn or pommel. The rear edges 3a and 4a of both wings have been indicated in FIG. 1C. As can be seen in FIGS. 1A and 1C a downwardly extending pin 7 has been provided within the component 1 at the rear end, which pin remains spaced apart from both wings 3 and 4. Said pin 7 is optional and may have been provided as intramedullary pin.

The mandibular component has been manufactured as one unity from titanium or tantalum, or from an alloy thereof, from PEEK or UHMWPE, or a composition of these or other materials, preferably manufactured using additional manufacturing and/or CNC robotic milling.

The mandibular component has been designed and manufactured in correspondence with the shape and condition of the ascending branch of the mandible of the patient in question, such that both wings fittingly abut the ascending branch of the maxilla, wherein the artificial condylar head 2 replaces the condylar head at the anatomic location, and the upper portion fittingly abuts the upper edge of the incisura semilunaris.

Depending on the circumstances, the medial wing may have been shaped differently, for instance be narrower, be L-shaped, or consist of several lips, for instance one lip near the condylar neck and one lip near the coronoid process.

Depending on the circumstances, the lateral wing may have been shaped differently, for instance be narrower, be L-shaped, or consist of several lips, for instance one lip near the condylar neck and one lip near the coronoid process.

The upper portion may further be interrupted in ridge direction, instead of continuous—as depicted in FIGS. 1A-D—.

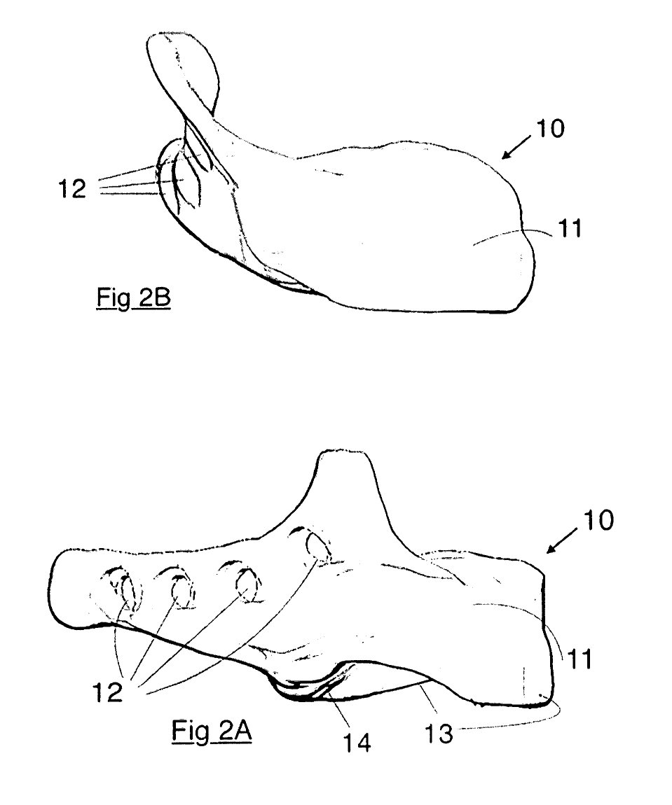

FIGS. 2A and 2B show a fossa component 10 for a TMJ prosthesis, which component is to be placed at the temporal side and capable of working together with the mandibular component 1. The fossa component 10 forms an elongated, downwardly opening socket 11 in which the artificial condylar head 2 is able to rotate and shift, as much in line with a natural jaw joint as possible. For attachment in the base of the patient's skull, bone screw holes 12 have been provided.

The fossa component may have been manufactured, optionally as one unity, from titanium or tantalum, or from an alloy thereof, from PEEK or UHMWPE, or a composition of these or other materials, preferably manufactured using additional manufacturing and/or CNC robotic milling. In one embodiment, the portion having the holes 12 has been made of metal, the portion of the socket 11 has been made of UHMWPE. The downwardly oriented bearing surface has a horizontal lip 14 at the front end and vertical lips 13 at the rear end and at the medial side in order to prevent luxation and reankylosis.

The fossa component has been designed and manufactured in correspondence with the shape and condition of the zygomatic arc and the glenoid cavity, including articular tubercle (tuberculum articularis), and in correspondence with the mandibular component.

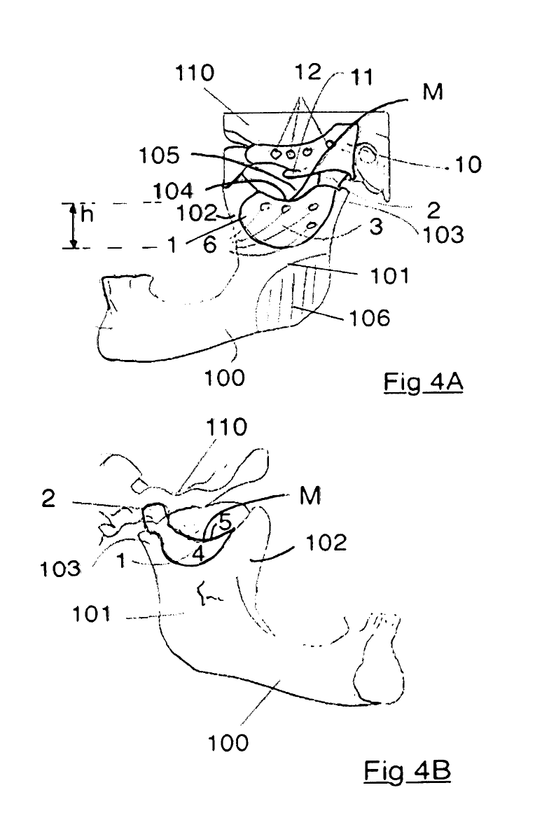

FIGS. 4A and 4B show a left half 100 of the mandible, including the ascending branch 101 (ramus mandibulae), the coronoid process 102 and the condylar process 103. When considered in a direction from rear to front (dorsal-ventral), the condylar process 103 merges into the coronoid process 102, via a concave ridge or upper edge 104 limiting the incisura semilunaris 105 in downward direction. Line 107 indicates the lower edge of the fossa infratemporalis.

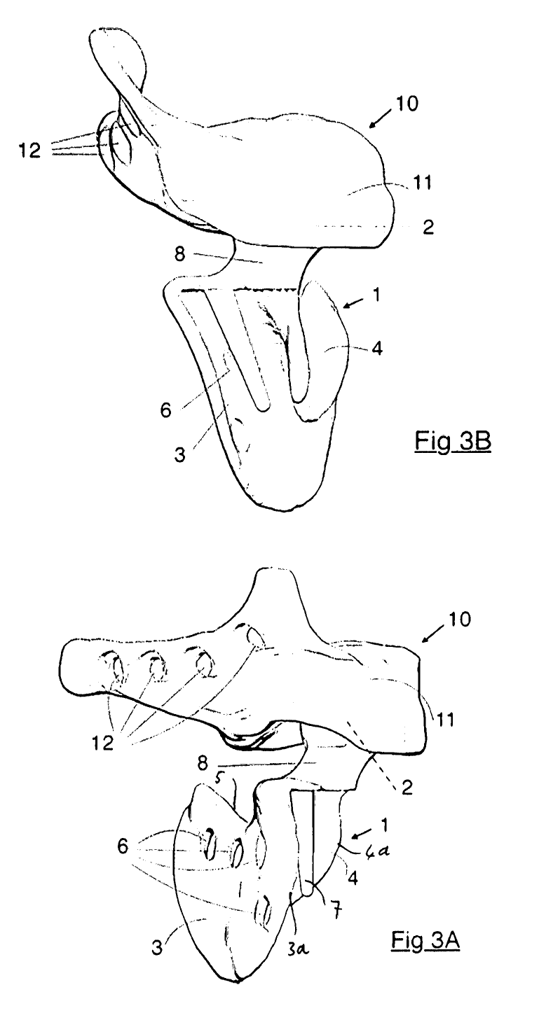

After preparatory actions, such as the surgical resection of the diseased condylar head (portion) and once the area has been made accessible by an incision in the area at the height of the temple (wherein the area of the jaw angle 106 is left virtually undisturbed), the mandibular component 1 is as it were placed like a saddle, the upper portion 5 resting on the concave ridge 104 and both wings 3 and 4 on either side, abutting both sides of the maxilla. As can be seen in FIG. 3A, the height h of the lateral wing 3 relative to the lowest point M of the ridge is approximately half the height or less of the ascending branch of the mandible 101, and the wing 3 remains above the buccal branch of the facial nerve. It can further be seen that in forward direction, the component 1 extends amply beyond the location M of the lowest point of the ridge 104. The “saddle” 1 is attached to the ascending branch 101, in its upper half, by means of bone screws in the holes 6 in the lateral wing 3.

The fossa component 10 is also placed and attached to the temporal bone 110 (os temporale) using bone screws in holes 12.

The saddle-shaped condylar head prosthesis according to the invention provides abutting contact with the bone on both sides of the ascending branch of the mandible, which enhances the stability and ingrowth of bone tissue. This considerably reduces the risk of the prosthesis getting dislodged.

The chewing forces are absorbed by the prosthesis 1 and transferred by it in a distributed manner to the mandible, namely to the condylar stump of the ascending branch, by pressure on the ridge and by transverse forces on the bone screws at the lateral side.

The relatively large surface of the lateral wing increases the freedom of choice in number and location of the holes for the bone screws.

In one embodiment, there is no medial wing in the mandibular component. The upper portion or ridge (which in that case is concave in cross-section, the cavity facing downwards) then extends onto/supports on the upper edge/ridge. The medial wing may also be short, when considered in downward direction, for instance in the form of an edge only just extending over the upper edge/ridge, so that in cross-section the look of a hook is realized.

The invention is/inventions are not at all limited to the embodiments discussed in the description and shown in the drawings. The above description has been included to illustrate the operation of preferred embodiments of the invention and not to limit the scope of the invention. Starting from the above explanation many variations that fall within the spirit and scope of the present invention will be evident to an expert. Variations of the parts described in the description and shown in the drawings are possible. They can be used individually in other embodiments of the invention(s). Parts of the various examples given can be combined together.

1-21. (canceled)

22. A prosthesis for the mandibular side of the temporomandibular joint, comprising a head part to replace the condylar head, in particular at the anatomic location, and an attachment plate for attachment to the mandible, wherein the attachment plate has been formed having a lateral portion that has been formed and is intended for abutting the lateral side of the ascending branch of the mandible, straight below the arcuate notch in the upper end of the ascending branch of the mandible, wherein the lateral portion has been provided with holes for attachment to the lateral side of the ascending branch of the mandible, wherein the attachment plate comprises an upper portion forming one unity with the lateral portion and formed and intended for abutting the concave upper edge of the arcuate notch in the upper end of the ascending branch of the mandible.

23. A prosthesis according to claim 22, wherein the attachment plate comprises a medial portion that forms one unity with the lateral portion and upper portion, and that has been formed and is intended for abutting the medial side of the ascending branch of the mandible, preferably straight below the arcuate notch in the upper end of the ascending branch of the mandible.

24. A prosthesis according to claim 22, wherein only the lateral portion has been provided with holes for attachment to the lateral side of the ascending branch of the mandible.

25. A prosthesis according to claim 22, wherein, in a direction parallel to the direction of projection of the head part, the lateral portion has a length that is smaller than its length in a direction transverse thereto.

26. A prosthesis according to claim 22, wherein the lateral portion is sickle-shaped.

27. A prosthesis according to claim 22, wherein the medial portion is sickle-shaped.

28. A prosthesis according to claim 22, wherein at least one of the lateral portion and the upper portion has been configured for in the placed condition extending in the ventral direction to beyond the lowest point of the arcuate upper edge, that means up into the area situated straight below the portion of the said upper edge which is ascending in the ventral direction in the direction of the coronoid process.

29. Prosthesis according to claim 23, wherein the medial portion has been configured for in the placed condition extending in the ventral direction to beyond the lowest point of the arcuate upper edge, that means up into the area situated straight below the portion of the said upper edge which is ascending in the ventral direction in the direction of the coronoid process.

30. A prosthesis according to claim 22, wherein at least one of the lateral portion and the upper portion has been formed and is intended to extend up to the coronoid branch.

31. A prosthesis according to claim 23, wherein the medial portion has been formed and is intended to extend up to the coronoid branch.

32. A prosthesis according to claim 22, wherein the head part and the attachment plate form one unity with each other.

33. A prosthesis according to claim 23, wherein the attachment plate has the look of a saddle, in particular the look of a hyperbolic paraboloid.

34. An assembly of a prosthesis according to claim 22 and a prosthesis for the fossa glenoidalis (glenoid cavity or glenoid fossa) as bearing part for the head part.

35. A method for replacing the condylar head in a patient by a condylar head prosthesis, wherein the shape of the edge of the arcuate notch in the upper end of the ascending branch of the mandible is defined and the prosthesis according to claim 22 is also designed and made on the basis thereof, after which the prosthesis is being placed on the ascending branch of the mandible in question.

36. A prosthesis for the mandibular side of a temporomandibular joint, comprising a head part to replace the condylar head, in particular at the anatomic location, and an attachment plate, wherein the attachment plate comprises a lateral portion and a medial portion that have been formed and are intended for abutting the lateral and medial sides, respectively, of the ascending branch of the mandible, straight below the arcuate notch in the upper end of the ascending branch of the mandible, and having an upper portion forming one unity therewith, being formed concave with the cavity facing upwards, when considered in a direction transverse to the lateral portion, and being formed concave, with the cavity facing downwards, when considered in a cross-section.

37. Prosthesis according to claim 36, wherein, in a direction parallel to the direction of projection of the head part, the lateral portion has a length that is smaller than its length in a direction transverse thereto.

38. Prosthesis according to claim 36, wherein at least one of the lateral portion and the medial portion is sickle-shaped.

39. Prosthesis according to claim 36, wherein at least one of the lateral portion, the upper portion and the medial portion has been configured for in the placed condition extending in the ventral direction to beyond the lowest point of the arcuate upper edge, that means up into the area situated straight below the portion of the said upper edge which is ascending in the ventral direction (in the direction of the coronoid process).

40. Prosthesis according to claim 36, wherein at least one of the lateral portion, the upper portion and the medial portion has been formed and is intended to extend up to the coronoid branch.

41. Prosthesis according to claim 36, wherein the head part and the attachment plate form one unity with each other and wherein the attachment plate has the look of a saddle, in particular the look of a hyperbolic paraboloid.
